(19) United States
(12) Patent Application Publication
(10) Pub. No.: US 2016/0067269 A1
(43) Pub. Date: Mar. 10, 2016

(54) Miltefosin Or Perifosin For Use In The Treatment of IBD

(71) Applicants: ACADEMISCH MEDISCH CENTRUM, Amsterdam (NL); ACADEMISCH ZIEKENHUIS LEIDEN H.O.D.N., Leiden (NL)

(72) Inventors: Gijs R Van Den Brink, Abcoude (NL); Auke Verhaar, Gouda (NL)

(73) Assignees: ACADEMISCH ZIEKENHUIS LEIDEN H.O.D.N., Leiden (NL); ACADEMISCH MEDISCH CENTRUM, Amsterdam (NL)

(21) Appl. No.: 14/782,815

(22) PCT Filed: Apr. 8, 2014
(86) PCT No.: PCT/EP2014/057042

§ 371 Date: Oct. 7, 2015

(30) Foreign Application Priority Data
Apr. 8, 2013 (EP) 13162702.8

(51) Int. Cl. A61K 31/685; A61K 45/06

(52) U.S. Cl. CPC A61K 31/685; A61K 45/06

(57) ABSTRACT

Miltefosin or perifosin for use in the treatment or prevention of inflammatory bowel disease (IBD).

### FIELD OF INVENTION

This invention relates generally to treatments for inflammatory bowel disease (IBD). In particular, this invention pertains to miltefosin (hexadecyl phosphocholine) or perifosin, (D-21266 or octadecyl 1,1-dimethyl-piperidino-4-yl phosphate), especially to pharmaceutical compositions containing miltefosin or perifosin for oral administration for treatment or prevention of IBD, especially of inflammation of the small intestine and colitis and including disease related extraintestinal manifestations.

### BACKGROUND OF THE INVENTION

During the last 50 years the incidence of people suffering from IBD has increased. Statistically one in a hundred people will suffer from one or more prolonged periods of IBD in their lifetime. During this period these patients will require treatment. Existing drugs all have downsides to their usage. Either they are very expensive or they cause severe side effects and none of them are guaranteed to work. Currently, aminosalicylates (antiinflammatory drugs) are used in the treatment of IBD to keep patients with mild to moderate disease in remission. Furthermore, corticosteroids (prednisone, methylprednisolone, hydrocortisone) are used to put a patient in remission but can only be used for a short time due to severe side effects. Patients can develop insensitivity to the therapy. Immunomodulators (Azathioprine, 6-MP, Cyclosporine, Tacrolimus, Methotrexate) are sometimes used to inhibit the immune response. Most are cytostatic drugs, inhibiting cell division and thereby decreasing white blood cells. Alternatively, antibiotics (Metronidazole, Ciprofloxacin) are sometimes used to decrease bacteria in the gut which is thought to decrease the immune response. Also biologic therapies using engineered proteins (Infliximab, Adalimumab, Certolizumab, Natalizumab) are used. These proteins target specific molecules (the cytokine TNFalpha or α4β7 integrin) and by doing so interfere with the anti-inflammatory response.

There is still a great need for new medicine.

### SUMMARY OF THE INVENTION

It was surprisingly and unexpectedly found according to one aspect of the present invention that miltefosin or perifosin are suitable for the treatment and prevention of IBD, especially of inflammation of the small intestine and colitis. Pharmaceutical administration of miltefosin or perifosin, for the treatment and/or prevention of IBD, especially of colitis, is neither described nor made obvious in the publications of the state of the art.

The invention therefore provides miltefosin or perifosin for use in the treatment or prevention of Inflammatory bowel disease (IBD).

In a further aspect, the invention provides a pharmaceutical formulation comprising a therapeutically effective amount of miltefosin or perifosin and optionally an an antiinflammatory drug, a corticosteroid or an antibiotic for use in the treatment or prevention of IBD.

In aspects of the invention, said pharmaceutical formulation of the invention is used for treatment of IBD by oral administration. Preferably, miltefosin or perifosin is administered orally at a daily dose of from about 10 mg to about 250 mg. Preferably, the total daily dose is about 20 mg to about 150 mg of miltefosin or perifosin. In a preferred embodiment the total daily dose is about 30 mg to about 100 mg of miltefosin or perifosin.

In another preferred embodiment, said treatment is by oral administration once, twice or three times weekly in total daily dose of about 10 mg, about 20 mg, about 50 mg, about 100 mg, or about 150 mg. Preferably, said treatment is by oral administration by multiple daily administrations in two or three equal portions. Preferably, said treatment is by oral administration which is continued over a period of from about 2 weeks to about 6 months. In another preferred embodiment, said treatment is a life long treatment.

In a further aspect, the invention provides a kit of parts for use in the treatment or prevention of IBD comprising the pharmaceutical formulation according to the invention and a separate composition comprising an antiinflammatory drug, a corticosteroid, an antibiotic or a compound selected from the group consisting of Infliximab, Adalimumab, Certolizumab, Vedolizumab and Natalizumab.

### BRIEF DESCRIPTION OF THE DRAWINGS

FIG. 1 shows the effect of miltefosine treatment on lymphocyte proliferation. Lymphocytes were stimulated with PHA (light colored bars) or SEB (dark colored bars). The effect was compared to dexamethasone treatment.

FIG. 2 is a FACS plot which shows the cells used to inject the SCID mice with to induce colitis. The vertical axis represents CD4 and the horizontal axis CD45RB. The black squares in the plot outline the cells used as CD45RBlow (left square) or CD45RBhigh (right square).

FIG. 3 is a graph which shows the average weight of each group of mice in an experimental mouse colitis model treated with miltefosine. The line with the round labels represents the control group which does not develop colitis and is not treated. The line with square labels represents the group of mice that do develop colitis but are not treated with miltefosine. The line with triangular labels represents the mice that develop colitis and are treated with miltefosine. Weight is given as a percentage of the weight of each mouse at the start of the experiment. The horizontal axis shows the days at which the mice were weighted.

FIG. 4A shows the actual average weight of each group at the end of the experiment.

FIG. 4B shows the average disease score of each group. The disease score is a cumulative score taking in account inflammation of the colon and stool consistency.

FIG. 5 shows a photo of a colon representative for each group.

FIG. 6A shows the actual average weight of each group at the end of the experiment.

FIG. 6B shows the average weight of the colon of each group per cm of colon.

FIG. 6C shows the average weight of the colon of each group.

FIG. 6D shows the average weight of the spleen of each group.

FIG. 7 shows a HE staining of a transverse section of the mouse colon wall representative for each group.

FIG. 8 shows the average pathology score of each group. The pathology score is a measure for the severity of inflammation in the colon. It takes in account epithelial cell hyperplasia, leukocyte infiltrations, loss of goblet cells, ulceration and crypt abscesses.

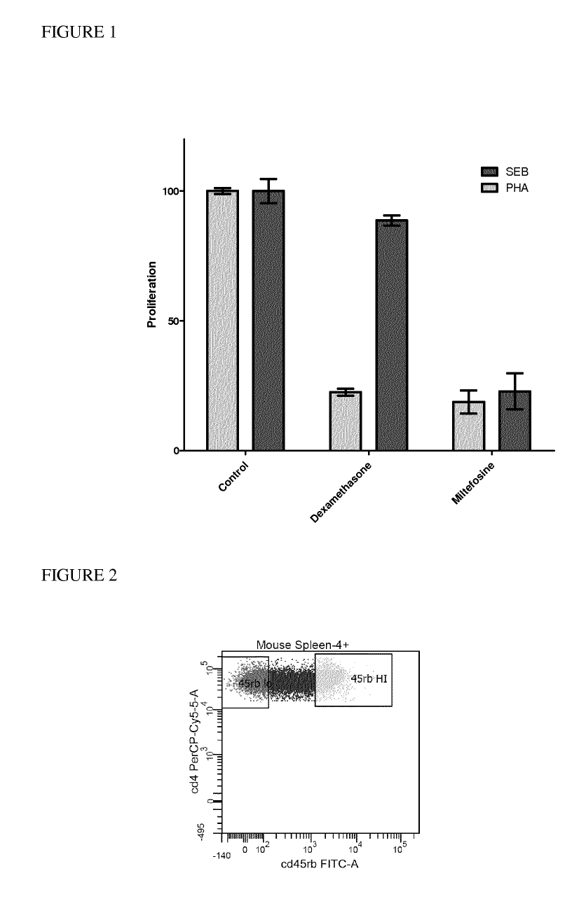

### DETAILED DESCRIPTION OF THE INVENTION

### Definitions

Herein, the term “treating” includes abrogating, substantially inhibiting, slowing or reversing the progression of a disease, substantially ameliorating clinical symptoms of a disease or substantially preventing the appearance of clinical symptoms of a disease.

The term “preventing” refers to barring a subject from acquiring a disorder or disease in the first place.

As used herein, the phrase “inflammatory bowel disease (IBD)” refers to a disorder or disease characterized by inflammatory activity in the GI tract. IBD includes, but is not limited to, Crohn's disease, ulcerative colitis, Johne's disease, Behçet's syndrome, collagenous colitis, diversion colitis, indeterminate colitis, microscopic colitis, infective colitis, ischaemic colitis, lymphocytic colitis, idiopathic inflammation of the small and/or proximal intestine, IBD-related diarrhea and closely related diseases and disorders of the gastrointestinal tract.

The term “administering”, as used herein, refers to a method for bringing the miltefosin or perifosin systemically in the body or into an area or a site in the GI tract that is affected by the IBD.

The term “therapeutically effective amount” refers to that amount of miltefosin or perifosin being administered, which will relieve to at least some extent one or more of the symptoms of the IBD being treated.

The present invention is based on the surprising finding that mice suffering from colitis treated with miltefosine had significantly reduced pathology scores compared to untreated controls. In addition, the inventors have also shown that mice could preventively treated with miltefosine.

### Embodiments

In accordance with one aspect of the present invention, a dosage plan is provided for treatment or prevention of IBD in man by the oral administration of the pharmaceutical composition. In the case of a suitable embodiment of the present invention, the following dosage plan is suitable for treatment or prevention of IBD in man by oral administration. Total dosage: 10 mg to 250 mg of miltefosin or perfosin, suitably 20 mg to 150 mg and especially 30 mg to 100 mg. Daily single or multiple dose: a total daily dose of 10 to 50 mg of active ingredient is administered suitably as a single daily dose. A dose of 50 to 250 mg of active ingredient and suitably of 50 to 150 mg of active ingredient is administered orally daily as a daily multiple dose, suitably as two doses per day (total daily dose of 100 mg of active ingredient) or as three doses per day (total daily dose of 150 mg). For most patients, this is a preferred embodiment. Certain patients cannot tolerate 3 times daily a dose of 50 mg. For these patients, 2 times daily a dose of 50 mg is preferred. From the point of view of compliance by the patients, a daily dose, divided into four to five doses, is generally regarded as the upper limit. For preventive purposes, however, it is also possible to administer the agent differently than divided into one to five doses per day.

In the case of a suitable embodiment, daily multiple doses of the same magnitude are administered (for example, 100 mg of active ingredient per day=2×50 mg of active ingredient per day or 150 mg of active ingredient per day=3×50 mg of active ingredient per day).

In accordance with a further aspect of the invention, a dosage plan for treatment or prevention of IBD in mammals other than man is made available by the oral administration of the pharmaceutical composition of the invention.

In accordance with a further aspect of the present invention, a combination of the pharmaceutical composition with an antiinflammatory drug, a corticosteroid or an antibiotic is made available for oral administration for treatment or prevention of IBD.

In a suitable embodiment of the invention, the pharmaceutical composition is administered in combination with an antiinflammatory drug, a corticosteroid or an antibiotic. The latter can be administered simultaneously or consecutively. The antiinflammatory drug, the corticosteroid and/or the antibiotic can be administered independently of one another. The antiinflammatory drug, the corticosteroid and/or the antibiotic can be contained either in the pharmaceutical compositon described or in a pharmaceutical formulation, which is independent thereof. In a preferred embodiment, said antiinflammatory drug comprises an antibody against tumor necrosis factor (TNF).

Suitable antiinflammatory drugs are aminosalicylates. Suitable corticosteroids include prednisone, methylprednisolone and hydrocortisone. Suitable immunomodulators include Azathioprine, 6-MP, Cyclosporine, Tacrolimus and Methotrexate. Suitable antibiotics are Metronidazole and Ciprofloxacin. In a preferred embodiment, also biologic therapies using engineered proteins (Infliximab, Adalimumab, Certolizumab, Vedolizumab, Natalizumab) are used. These proteins target specific molecules (the cytokine TNFalpha or α4β7 integrin)

The solid, oral pharmaceutical compositions are suitably useful for treatment or prevention of IBD.

### EXAMPLE

### Miltefosine

To study the different molecular signaling pathways that are activated when lymphocytes are stimulated, we used multiple pharmaceutical inhibitors. One of these was Miltefosine, an ether lipid drug developed for the topical treatment of cutaneous breast cancer metastasis^{[1]} but best known for the treatment of leishmaniasis^{[2]}, a parasitic infectious disease^{[3]}.

How the drug works exactly is still under intense investigation. Miltefosine has great similarity to the phospholipids that make up the cellular membrane. It has been shown that it incorporates in the outer layer of the membrane and is actively internalized by a specific transporter complex^{[4]}. This complex is present in sterol dense areas on the membrane, known as lipid rafts^{[5]}. These lipid rafts are important signaling platforms where receptors and kinases accumulate to transfer signals from outside the cell^{[6]}.

### Inhibitory Effect of Miltefosine on Lymphocyte Proliferation

Peripheral blood lymphocytes were isolated from buffycoat (Sanquin) by ficoll/percoll density gradient centrifugation. In short the buffy coat was placed on top of a layer ficoll and spinned to separate peripheral blood mononuclear cells. These were washed in RPMI1640 medium and brought on top of hyperosmotic percoll solution (48.5% Percoll, 41.5% H₂O, 10% 1.6M NaCl) to separate monocytes from lymphocytes.

The lymphocytes were treated with miltefosine (61 μM) or dexamethasone (1 μM) and stimulated with 10 μg/ml phytohematoglutinin (PHA) or 100 ng/ml staphylococcal enterotoxin B (SEB) for 24 hours. Then tritiated thymidine (10 μCi/ml) was added to each well and left overnight. Next morning incorporation was measured using a microbeta counter.

The graph shows proliferation of lymphocytes as measured by thymidine incorporation assay. Values were corrected to the untreated control conditions. Miltefosine treatment was compared to treatment with the synthetic glucocorticoid dexamethasone, a very powerful immunosuppressive agent. The lymphocytes were stimulated with the mitogenic lectin PHA or with the superantigen SEB. The latter is known to cause steroid resistant proliferation in vitro. In both cases miltefosine successfully suppresses proliferation.

### Miltefosine in an Experimental Mouse Colitis Model

To study the application of miltefosine as an anti-inflammatory drug, miltefosine was tested in the experimental colitis transfer model. In this model SCID mice, lacking T and B lymphocytes, are injected with only effector T lymphocytes (CD4⁺CD45RB^{High}) omitting regulatory T lymphocytes (CD4⁺CD45RB^{Low}). The absence of the letter causes, over a period of two months, the development of chronic inflammation of the colon.

### Method

Mouse CD4 positive lymphocytes were isolated from the spleen of normal mice (Balb/c OlaHsd). The spleen was removed, forced through a cell strainer to obtain a single cell suspension. Erythrocytes were lysed by adding water, quickly followed by PBS. The remaining cells were enriched in CD4 positive cells by negative depletion using magnetic beads specific for B cell (anti-B220), macrophages (anti-Mac-1) and cytotoxic T lymphocytes (anti-CD8). The remaining cell were washed and labeled for CD4 (CD4-PerCP-Cy5) and CD45RB (CD45RB-FITC). Using a FACS sorter the cell were separated in CD4 cells expressing low levels of CD45RB (CD45RB^{Low}) and high levels of CD45RB (CD45RB^{High}).

To induce colitis, SCID mice (C.B-17/lcrHsd-Prkcd) were injected with 5·10⁵ CD45RB^{High} cells. The control group which did not develop colitis was injected with 5·10⁵ CD45RB^{High} cells plus 2.5·.10⁵ CD45RB^{Low} cells.

### Study Design

The mice were divided in three groups, each group containing 10 mice. The first group was injected with both CD45RB^{High} and CD45RB^{Low} and served as a negative control group which did not develop colitis. Group two and three both received only CD45RB^{High} cells and developed colitis. Only group three received miltefosine (50 mg/kg) via oral gavage. Treatment started two weeks after the start of the experiment and was given twice a week until the end of the experiment. The mice were weighted three times a week during the entire duration of the experiment.

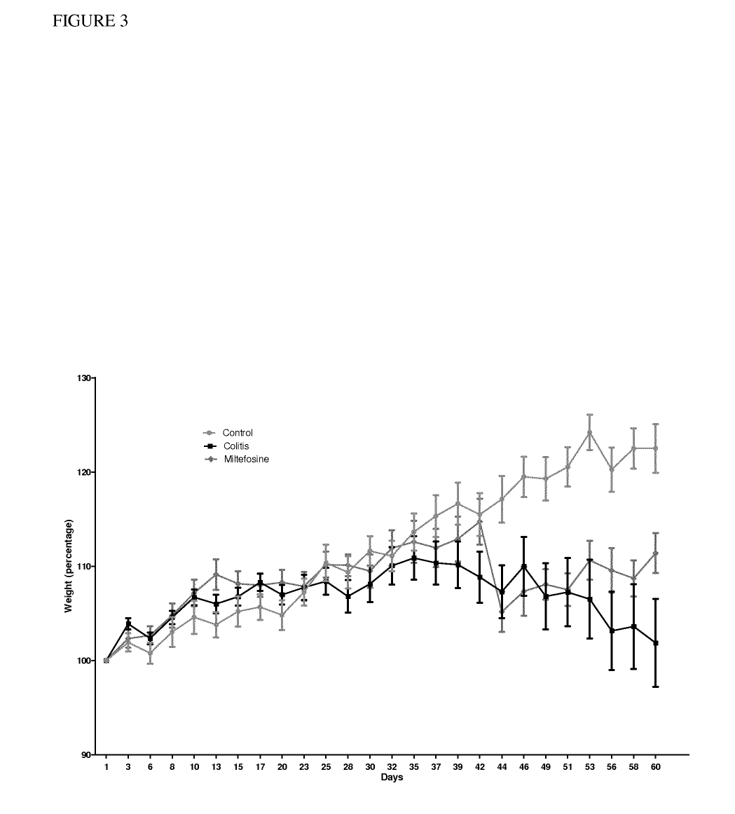

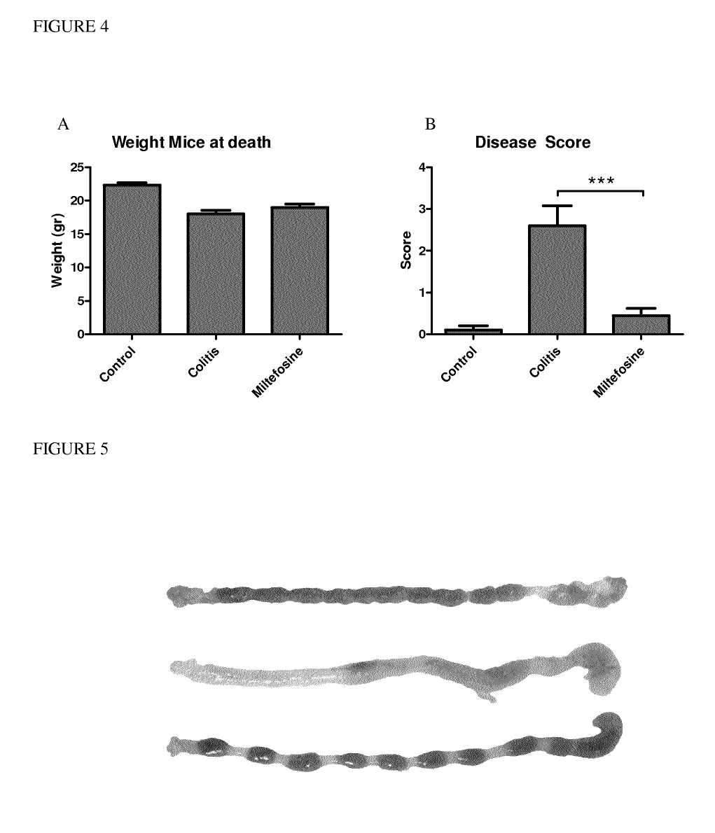

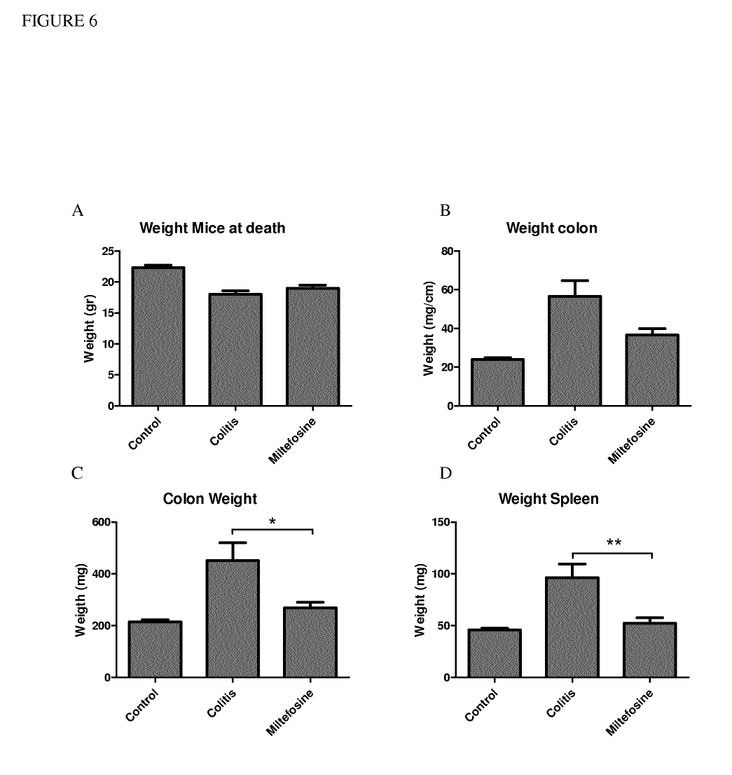

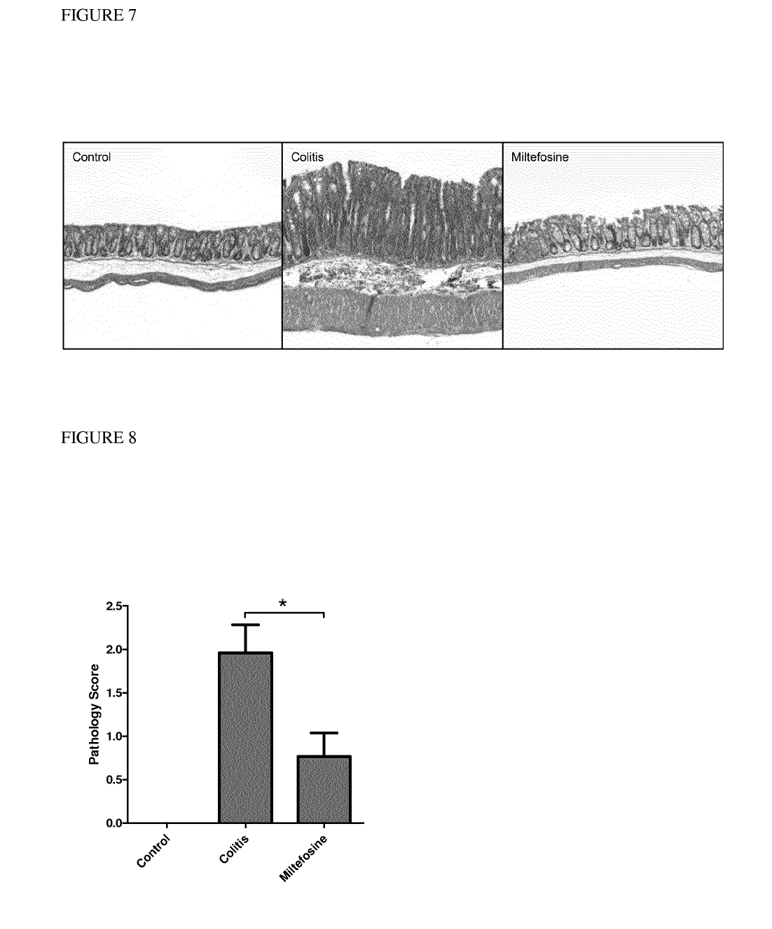

The lines in FIG. 3 represent the average weight of each group with the grey line with the circular labels (balls) as the negative control group. The weight of the mice in this group increased steadily over the entire 60 days of the experiment. The black line with the square labels (squares) is the positive control group. These mice develop inflammation of the colon. The group treated with miltefosine. Group 4 is represented by the dark line with the triangular labels.(rhombus). Till day 42 the weight of these mice corresponded with the negative control group. But for unknown reasons they lost ten percent of their weight in two days time after which they continued to gain weight. During this time the mice looked completely healthy and continued to do so.

The average weight of the different groups at the end of the experiment does not differ significantly. To assess the overall condition of each mice, inflammation and oedema of the colon was scored combined with stool consistency. The sum of the scores resulted in the disease score. Compared to the positive control group the mice that were preventively treated had a significantly lower disease score. This was also apparent in their behavior and appearance. The mice were more active and their fur was smooth.

Another way to quantify inflammation is to score length and weight of the colon. As inflammation increases, the length decreases. Partially due to the decrease in length but also because of oedema, the weight of the colon increases. Treatment with miltefosine significantly improved the weight of the colon. Inflammation can cause the spleen to enlarge and increase in weight. This increase was significantly reduced when treated with miltefosine.

### Pathology

Part of the colon of each mouse was embedded in paraffin, cut and stained with hematoxylin and eosin (HE). The stained coupes were scored blindly by a pathologist and the result summarized in FIG. 8. The pathology score takes in account epithelial cell hyperplasia, leukocyte infiltrations, loss of goblet cells, ulceration and cryptabscesses.

Treatment with miltefosine significantly reduced the pathology score. There is a clear decrease in crypt length and thickness of the submucosa and muscle layer.

### Conclusion

Here we investigated the possible application of miltefosine as an oral drug for the treatment of inflammatory bowel diseases in an experimental mouse colitis model. Treatment started two weeks after the start of the experiment and was given twice a week until the end of the experiment. The amount of miltefosine per treatment is 50 mg/kg. Although this is an accepted concentration^{[11]} it has been reported that this concentration can affect bodyweight negatively^{[12]}. Normally bodyweight is an excellent parameter to measure disease development. But in this experiment the bodyweight doesn't seem to correlate with the inflammatory condition of the mice. This could be due to the anomaly on day 42 but a decrease in the dosage of miltefosine might improve the overall bodyweight scores. Treatment with high dosages of miltefosine is known to cause nausea and vomiting in human patients and although mice lack the ability to throw up, treatment may reduce their appetite.

Administering oral dosages of miltefosine twice a week, reduced inflammation and improved the overall condition of the mice. With the exception of the weight curve and based on the pathology score and the disease score it can be concluded that this group was successfully treated.

### REFERENCES

1. Eibl H, Unger C (1990) Hexadecylphosphocholine: a new and selective antitumor drug. Cancer Treat Rev 17: 233-242.
2. Sundar S, Rosenkaimer F, Makharia M K, Goyal A K, Mandal A K, Voss A, Hilgard P, Murray H W (1998) Trial of oral miltefosine for visceral IBD. Lancet 352: 1821-1823.
3. Chappuis F, Sundar S, Hailu A, Ghalib H, Rijal S, Peeling R W, Alvar J, Boelaert M (2007) Visceral IBD: what are the needs for diagnosis, treatment and control? Nat Rev Microbiol 5: 873-882.
4. Menez C, Buyse M, Farinotti R, Barratt G (2007) Inward translocation of the phospholipid analogue miltefosine across Caco-2 cell membranes exhibits characteristics of a carrier-mediated process. Lipids 42: 229-240.
5. Saint-Pierre-Chazalet M, Ben B M, Le M L, Bories C, Rakotomanga M, Loiseau P M (2009) Membrane sterol depletion impairs miltefosine action in wild-type and miltefosine-resistant Leishmania donovani promastigotes. J Antimicrob Chemother 64: 993-1001.
6. Lingwood D, Simons K (2010) Lipid rafts as a membrane-organizing principle. Science 327: 46-50.
8. Weller K, Artuc M, Jennings G, Friedrichson T, Guhl S, dos Santos RV, Sunder C, Zuberbier T, Maurer M (2009) Miltefosine inhibits human mast cell activation and mediator release both in vitro and in vivo. J Invest Dermatol 129: 496-498.
9. Hartmann K, Siebenhaar F, Belloni B, Brockow K, Eben R, Hartmann B, Rueff F, Schoepke N, Staubach P, Weber A, Maurer M (2010) Effects of topical treatment with the raft modulator miltefosine and clobetasol in cutaneous mastocytosis: a randomized, double-blind, placebo-controlled trial. Br J Dermatol 162: 185-190.
10. Dolle S, Hoser D, Rasche C, Loddenkemper C, Maurer M, Zuberbier T, Worm M (2010) Long-term reduction in local inflammation by a lipid raft molecule in atopic dermatitis. Allergy 65: 1158-1165.
11. Baumer W, Wlaz P, Jennings G, Rundfeldt C (2010) The putative lipid raft modulator miltefosine displays immunomodulatory action in T-cell dependent dermal inflammation models. Eur J Pharmacol 628: 226-232.
12. Aguiar M G, Silva D L, Nunan F A, Nunan E A, Fernandes A P, Ferreira L A (2009) Combined topical paromomycin and oral miltefosine treatment of mice experimentally infected with Leishmania (Leishmania) major leads to reduction in both lesion size and systemic parasite burdens. J Antimicrob Chemother 64: 1234-1240.

1. Miltefosin or perifosin for use in the treatment or prevention of inflammatory bowel disease (IBD).

2. A pharmaceutical formulation comprising a therapeutically effective amount of miltefosin or perifosin and optionally an antiinflammatory drug, a corticosteroid or an antibiotic for use in the treatment or prevention of IBD.

3. The pharmaceutical formulation of claim 2, wherein treatment is by oral administration.

4. The pharmaceutical formulation of claim 3, wherein miltefosin or perifosin is administered orally at a daily dose of from about 10 to about 250 mg.

5. The pharmaceutical formulation of claim 3, wherein the total daily dose is about 20 to about 150 mg of miltefosin or perifosin.

6. The pharmaceutical formulation of claim 3, wherein the total daily dose is about 30 to about 100 mg of miltefosin or perifosin.

7. The pharmaceutical formulation of claim 3, wherein treatment is by oral administration once, twice or three times weekly in total daily dose of about 10 mg, about 20 mg about 50 mg, about 100 mg, or about 150 mg.

8. The pharmaceutical formulation of claim 3, wherein treatment is by oral administration by multiple daily administrations in two or three equal portions.

9. The pharmaceutical formulation of claim 3, wherein the oral administration is continued over a period of from about 2 weeks to about 6 months or lifelong.

10. A kit comprising the pharmaceutical formulation according to claim 3 and a separate pharmaceutical composition comprising an antiinflammatory drug, a corticosteroid, an antibiotic or a compound selected from the group consisting of Infliximab, Adalimumab, Certolizumab, Natalizumab for use in the treatment or prevention of inflammatory bowel disease (IBD).

11. The pharmaceutical composition according to claim 2, wherein said inflammatory bowel disease is colitis.
